(19) United States
(12) Patent Application Publication
(10) Pub. No.: US 2020/0333342 A1
(43) Pub. Date: Oct. 22, 2020

(54) BREAST CANCER DETECTION KIT USING SALIVA AND BREAST CANCER DETECTION METHOD USING SAME, AND FERTILE WINDOW DETECTION KIT USING SALIVA AND FERTILE WINDOW DETECTION METHOD USING SAME

(71) Applicant: HENRY TECHNOLOGY CO., LTD., Busan (KR)

(72) Inventor: Jong Ha LEE, Daegu (KR)

(73) Assignee: HENRY TECHNOLOGY CO., LTD., Busan (KR)

(21) Appl. No.: 16/756,370

(22) PCT Filed: Oct. 11, 2018
(86) PCT No.: PCT/KR2018/011994

§ 371 Date: Apr. 15, 2020

(30) Foreign Application Priority Data
Oct. 16, 2017 (KR) 10-2017-0134264
Oct. 16, 2017 (KR) 10-2017-0134278

(51) Int. Cl. G01N 33/574; G01N 1/28; G01N 21/78; G01N 33/76; G01N 33/543

(52) U.S. Cl. CPC G01N 33/57415; G01N 33/57488; G01N 1/28; G01N 2333/59; G01N 33/76; G01N 33/54366; G01N 21/78

(57) ABSTRACT

A breast cancer detection kit using saliva and a breast cancer detection method using the kit, which are presented in the present invention, can detect breast cancer more accurately than a conventional breast cancer detection technique. In addition, a fertile window detection kit using saliva and a fertile window detection method using the kit, which are presented in the present invention, can resolve inconveniences of limitations on time and place in a conventional technology for confirming whether there is a fertile window through urine, and can solve problems, of a conventional technique for determining the structure of saliva through observation, in which saliva takes a certain amount of time to dry and the observation, itself, of spit can arouse visual discomfort, in order to confirm the salt crystals of saliva or the structural shape of saliva.

### TECHNICAL FIELD

The present invention relates to a breast cancer detection kit and a breast cancer detection method using the kit, and more specifically to a breast cancer detection kit using saliva and a breast cancer detection method using the kit. Further, the present invention relates to a fertile window detection kit and a fertile window detection method using the kit, and more specifically to a fertile window detection kit using saliva and a fertile window detection method using the kit.

### BACKGROUND ART

As the living environment of the modern society changes, the incidence of various diseases is increasing, and in particular, the adult diseases such as heart disease, obesity, etc. are increasing. Among women, the incidence of breast cancer is increasing more rapidly than in the past. With such a high rise in the incidence of breast cancer, various techniques for preventing and treating this disease are emerging.

As a general method for examining breast cancer, there is mammography. FIG. 1 is a view showing a test result of the conventional mammography. The mammography reads a tumor having such a size of about 1 cm on average that it frequently detects breast cancer at a stage B, thus having a limit as an early diagnostic equipment. Further, the construction for the equipment costs about two to three hundred million won (KRW) and there is a disadvantage of being exposed to radiation. In addition, the mammography has a problem in that it gives an unpleasant feeling and pains to women during its screening for breast cancer. When it comes to diagnosing breast cancer, breast ultrasonography has been often used along with the mammography. FIG. 2 is a view showing a test result of the conventional breast ultrasonography. In Korea, where there many young patients with breast cancer and many women with dense breast tissues, the breast ultrasonography has grown much in importance due to the characteristics thereof. Recently, as ultrasonic waves have a higher resolution little by little, medical specialists have preferred an examination using ultrasonic waves more than before and have been also able to make a much higher quality diagnosis than before through the ultrasonography. However, despite the advantages of being a real-time examination, the ultrasonography shows a large difference in diagnosis results depending on the ultrasound equipment, and shows a lot of difference depending on the experience and knowledge of inspectors. Thus, despite its importance, the reality is that the ultrasonography is not recognized as a standard and objective test method in group tests, etc. In addition, since doctors have to confirm with their own eyes if there are any lesions one by one while directly taking pictures with ultrasonic waves. Thus, the general hospitals with a large number of patients have a problem in that a rate of wrong diagnosis gets higher with an increase in doctor's fatigue.

In addition to the mammography and the ultrasonography described above, there is a technique for predicting cancer using DNA as a way for detecting cancer. This is a method for predicting cancer by confirming a mutation of breast cancer genes. Specifically, such method may be applied to predicting cancer by detecting the cancer gene information (DNA) present in saliva to analyze the genes of cells, that is, by confirming the genes using saliva. As the prior art related thereto, there are the registered patent No. 10-1142452 (invention title: Method for Detecting Methylation of Breast Cancer Specific Methylation Marker Gene for Breast Cancer Diagnosis) and the like.

Meanwhile, ovulation means that a mature ovarian follicle bursts to release follicular fluid and an egg under the influence of hormone, and an ovulation day refers to the day which is 14 days before the expected onset of menstruation in a following month. A fertile window means a period in which a woman can conceive a baby, and it is known that such period ranges from four days before, the very day of and two days after an ovulation day and a subsequent period is an infertile period. FIG. 3 is a view for explaining a woman's menstrual cycle. It may be confirmed according to FIG. 3 that the fertile window ranges from Days 11 to 16 based on Day 14, i.e., the ovulation day. As a symptom of the ovulation period, there is a change in basic body temperatures, which occurs due to the influence of progesterone (leuteinizing hormone) secreted during the ovulation period.

To confirm a child-bearing period, the ovulation period is confirmed based the change in body temperatures as mentioned above. However, such period is also calculated by various methods such as Ogino's method for ovulation cycle, a natural ovulatory cycle method, etc. In addition, there are other methods using urine or saliva to confirm whether there is an ovulation period based on a body change during the ovulation period.

FIG. 4 is a view showing a conventional ovulation tester using urine, in which a test strip (stick) is soaked in urine to confirm whether an ovulation strip is positive, that is, to confirm if a luteinizing hormone (LH), i.e., a hormone for making corpora lutea, is detected. The luteinizing hormone (LH) is released from a pituitary gland of hypothalamus and results in the rupture of the ovarian follicle, thus causing corpora lutea to be released therefrom. The luteinizing hormone (LH) is released in a large amount at a point of time when the ovarian follicle is swollen and about to burst, and thus the large amount of the LH is released at a time point of ovulation. The luteinizing hormone (LH) released above is secreted into blood. If there is a large amount of secretion, the luteinizing hormone (LH) is detected even from urine. Then, information displayed on the ovulation tester is set to vary depending on how much the luteinizing hormone (LH) is detected from the urine and thus to confirm whether there is an ovulation period.

As such, there have been already various ongoing studies on a fertile window detection method. However, a conventional technique for confirming whether there is a fertile window through urine has limitations on time and place because it is required to take the trouble to go and urinate in a certain place in order to confirm whether there is a fertile window. To address the inconvenience of such technique for determining the fertile window using urine, there is a need to develop a technique for determining the fertile window using saliva without any constraints in time and place.

FIG. 5 is a view showing a conventional portable ovulation tester using saliva. Unlike the ovulation tester using urine in FIG. 4, FIG. 5 shows an ovulation tester using saliva (spit) based on a technique for classifying an ovulation cycle into an infertile period, an intermediate period and a fertile period with one drop of saliva. A rate of saliva secretion is known to have a statistically significant correlation between proliferative and ovulation periods, proliferative and luteal periods, and ovulation and luteal periods during a menstrual cycle. In particular, the technique uses the characteristics of saliva, in which a spit viscosity increases as a body temperature rises in the fertile window of the menstrual cycle. As an example, a product called “Bebless” in current use works in such a way that a film is sequentially stained with saliva, put into the product, and fixed to a mobile phone camera to display information on a liquid crystal display to confirm whether there is an ovulation. This product is configured to take a picture of the salt crystals of saliva with a smart phone to make an analysis accordingly because the salt crystals of saliva vary depending on a menstrual cycle. As shown in FIG. 5(a), a product is configured to determine whether there is an ovulation day in such a way that spit is slightly applied onto a lens to dry, and the structure of the spit is figured out through a holder. According to such method, this product is configured to determine the ovulation day, if the structure of spit looks interlaced like frost. As shown in FIG. 5(b), a product is a portable ovulation tester configured to accurately diagnose a woman's irregular infertile period and fertile period by using saliva within five minutes, because the structure of spit changes during an ovulation period, i.e., a child-bearing period.

FIG. 6 is a view for explaining a detection principle of a conventional portable ovulation tester using saliva, and shows the result of measuring a menstrual cycle by using the product as shown in FIG. 5(b). FIG. 6 shows the structure of spit for each period, in which (a) takes on a dot shape without a certain pattern, referring to an infertile period without ovulation; (b) takes on a shape of many gravels along with ferny leave patterns, referring to an intermediate period, i.e., an ovulation latent period, which is three days before the onset of ovulation and thus means a child-bearing period considering a maximum survival time of sperms; and (c) takes on a lattice pattern of plant leaves, referring to an ovulation period, which falls on two days of actual ovulation with the highest chance to conceive a baby.

As described above, the technique for using saliva is to observe a change in the structure of spit due to the viscosity of saliva. As a prior art related thereto, there are the registered patent No. 10-0860681 (invention title: Device for determining an ovulation period and portable wireless communication terminal using the same) and the like.

However, a conventional technique for determining the structure of saliva through observation has a problem in that saliva takes a certain amount of time to dry and the observation, itself, of spit can arouse visual discomfort to confirm the salt crystals of saliva or the structural shape of saliva.

Saliva is an important biological fluid which provides various functions including lubrication for speech, digestion of food and protection from microorganisms, and saliva consists of 99% of water along with minerals, mucus, electrolytes, enzymes, enzyme inhibitors, growth factors, cytokines, immunoglobulins and other glucoproteins. Saliva, which consists of various components, may be used to monitor a current clinical state and predict systemic diseases according to the filtration of blood reflecting the physiological conditions of a body. When comparing a diagnosis using saliva with a diagnosis using blood, the diagnosis using saliva provides a distinct advantage for the purpose of diagnosis or study, is advantageous in terms of cost, and has a great advantage of being a non-invasive process before everything. Thus, there has been an increase in studies using saliva.

Recently, there has been developed a method for classifying into women without any abnormalities in breast, women with benign tumors therein and women with malignant tumors therein by analyzing 49 proteins in such saliva, or the like.

There has been already an ongoing study on a method for detecting cancer using saliva and an analysis has been made on various proteins, etc. within the saliva. Such method has a high probability of detection, but needs a certain equipment capable of analysis, which works on a large scale with high costs of analysis. Thus, there is a problem in that it is difficult to popularize such method. To solve the problems above, there is a need to develop a technique for detecting breast cancer with a kit for detecting proteins, in which the kit may detect breast cancer by using saliva, may be confirmed with naked eyes without a particular need for a separate equipment, may be easily portable, and thus may be popularized.

Meanwhile, there is a need to develop a technique for detecting a fertile window by using various components, i.e., a hormone contained in saliva without being constrained by time and space, while addressing the problems of a conventional technique which confirms a fertile window by observing the structure of saliva, at the same time.

### DETAILED DESCRIPTION OF THE INVENTION

### Technical Problem

The present invention is proposed to solve the aforesaid problems of the conventional methods and an object of the present invention is to provide a breast cancer detection kit using saliva and a breast cancer detection method using the kit, wherein the breast cancer detection kit is configured to include: a collection module configured to collect saliva of a user; a detection module configured to detect a breast cancer marker from the saliva after removing mucus glycoproteins from the collected saliva as a pre-treatment process for detecting the breast cancer marker from the saliva collected from the collection module; and an analysis module configured to analyze a presence of breast cancer by using an immune reaction of the breast cancer marker detected from the detection module, wherein the breast cancer marker detected from the saliva includes at least one of Lysine (409.2312 m/z), Threonine (131.1174 m/z), Glutamic acid, Tyrosine, Piperideine, Valine, and Glycine (437.7442 m/z), wherein a process for removing mucus glycoproteins from the collected saliva is performed as a pre-treatment process for detecting the breast cancer marker, and wherein the analysis module is configured to analyze the presence of breast cancer through a reaction with an antibody which performs an antigen-antibody reaction with the breast cancer marker, thereby more accurately detecting the presence of breast cancer than a conventional technique for detecting breast cancer.

Further, another object of the present invention is to provide a breast cancer detection kit using saliva and a breast cancer detection method using the kit, wherein the breast cancer marker sequentially reacts with an antibody, promotes a color formation and amplifies the promoted color formation, wherein the breast cancer detection kit is configured to include a display module which displays information to confirm the presence of breast cancer based on the analysis result of the analysis module, wherein the presence of breast cancer may be confirmed with naked eyes without a particular need for a separate equipment, and wherein the breast cancer detection kit costs less than a conventional technique for detecting breast cancer, may be easily portable, may be popularized, and may detect a woman's breast cancer at an early stage to be treated.

Meanwhile, the present invention is proposed to solve the aforesaid problems of the conventional methods and an object of the present invention is to provide a fertile window detection kit using saliva and a fertile window detection method using the kit, wherein the fertile window detection kit is configured to include: a collection module configured to collect saliva of a user; a detection module configured to detect a hormone from the saliva collected from the collection module; and an analysis module configured to analyze whether there is a fertile window by using an immune reaction of the hormone detected from the detection module, and wherein the analysis module is configured to analyze whether there is a fertile window through an antigen-antibody reaction as the antigen-antibody reaction between the hormone and the antibody, thereby resolving inconveniences of limitations on time and place in a conventional technology for confirming whether there is a fertile window through urine, and solving problems of the conventional technique for determining the structure of saliva through observation, in which saliva takes a certain amount of time to dry and the observation, itself, of spit can arouse visual discomfort, in order to confirm the salt crystals of saliva or the structural shape of saliva.

Further, another object of the present invention is to provide a fertile window detection kit using saliva and a fertile window detection method using the kit, wherein the hormone collected from the collection module includes a luteinizing hormone and a process for removing mucus to purify the saliva after decomposing the glucoproteins within the collected saliva is performed as a pre-treatment process for detecting the hormone, which removes mucus glycoproteins from the collected saliva, and a process for changing a pH value of the collected saliva into pH7 and adding a buffer solution to mix the buffer solution with the collected saliva is performed in order to remove microorganisms and facilitate an antigen-antibody reaction, thereby increasing the accuracy of detecting and analyzing the hormone, and more accurately detecting whether there is a fertile window than a conventional technique for detecting the fertile window.

In addition, another object of the present invention is to provide a fertile window detection kit using saliva and a fertile window detection method using the kit, wherein a hormone sequentially reacts with an antibody, promotes a color formation and amplifies the promoted color formation, wherein the fertile window detection kit is configured to include a display module which displays information to confirm whether there is a fertile window based on the analysis result of the analysis module, wherein whether there is a fertile window may be confirmed with naked eyes without a particular need for a separate equipment and may be quickly confirmed without a need for additional time for saliva to get dry compared to a conventional technique for detecting a fertile window, and wherein the fertile window detection kit may be small-sized, may be easily portable and may be popularized.

### Technical Solution

In order to achieve the above objects, a breast cancer detection kit using saliva is configured to include: a collection module configured to collect saliva of a user; a detection module configured to detect a breast cancer marker from the saliva after removing mucus glycoproteins from the collected saliva as a pre-treatment process for detecting the breast cancer marker from the saliva collected from the collection module; an analysis module configured to analyze a presence of breast cancer by using an immune reaction of the breast cancer marker detected from the detection module; and a display module configured to display information to confirm the presence of breast cancer based on the analysis result of the analysis module.

Preferably, the breast cancer marker may include at least one of Lysine (409.2312 m/z), Threonine (131.1174 m/z), Glutamic acid, Tyrosine, Piperideine, Valine, and Glycine (437.7442 m/z).
Preferably, the mucus glycoproteins may include mucosal glycoproteins.
Preferably, the removal of the mucus glycoproteins may be obtained by decomposing glucoproteins within the collected saliva.
More preferably, mucus may be removed to purify the saliva after decomposing the glucoproteins.

Preferably, the detection module may be configured to remove microorganisms from the collected saliva.

Preferably, the detection module may be configured to change a pH value of the collected saliva into pH7.
Preferably, the detection module may be configured to add a buffer solution to mix the buffer solution with the collected saliva.

Preferably, the analysis module may be configured to analyze the presence of breast cancer through an antigen-antibody reaction, in which an immune reaction of the analysis module is the antigen-antibody reaction between the breast cancer marker detected from the collected saliva and an antibody.

More preferably, the breast cancer marker may sequentially react with the antibody, promote a color formation, and amplify the promoted color formation.

Further, in order to achieve the above objects, a breast cancer detection method using a breast cancer detection kit using saliva is configured to include: (a) collecting, by a collection module, saliva of a user; (b) detecting, by a detection module, a breast cancer marker from the saliva after removing mucus glycoproteins from the collected saliva as a pre-treatment process for detecting the breast cancer marker from the saliva collected from the collection module of step (a); (c) analyzing, by an analysis module, a presence of breast cancer by using an immune reaction of the breast cancer marker detected from the detection module of step (b); and (d) displaying, by a display module, information to confirm the presence of breast cancer based on the analysis result of the analysis module of step (c).

Preferably, the breast cancer marker may include at least one of Lysine (409.2312 m/z), Threonine (131.1174 m/z), Glutamic acid, Tyrosine, Piperideine, Valine, and Glycine (437.7442 m/z).

Preferably, the mucus glycoproteins may include mucosal glycoproteins.

Preferably, the removal of the mucus glycoproteins may be obtained by decomposing glucoproteins within the collected saliva.

More preferably, mucus may be removed to purify the saliva after decomposing the glucoproteins.

Preferably, the detection module may be configured to remove microorganisms from the collected saliva.

Preferably, the detection module may be configured to change a pH value of the collected saliva into pH7.

Preferably, the detection module may be configured to add a buffer solution to mix the buffer solution with the collected saliva.

Preferably, the analysis module may be configured to analyze the presence of breast cancer through an antigen-antibody reaction, in which an immune reaction of the analysis module is the antigen-antibody reaction between the breast cancer marker detected from the collected saliva and an antibody.

More preferably, the breast cancer marker may sequentially react with the antibody, promote a color formation, and amplify the promoted color formation.

Meanwhile, in order to achieve the above objects, a fertile window detection kit using saliva is configured to include: a collection module configured to collect saliva of a user; a detection module configured to detect a hormone from the saliva after removing mucus glycoproteins from the collected saliva as a pre-treatment process for detecting the hormone from the saliva collected from the collection module; an analysis module configured to analyze whether there is a fertile window by using an immune reaction of the hormone detected from the detection module; and a display module configured to display information to confirm whether there is a fertile window based on the analysis result of the analysis module.

Preferably, the hormone may include a luteinizing hormone.

Preferably, the mucus glycoproteins may include mucosal glycoproteins.

Preferably, the removal of the mucus glycoproteins may be obtained by decomposing glucoproteins within the collected saliva.

More preferably, mucus may be removed to purify the saliva after decomposing the glucoproteins.

Preferably, the detection module may be configured to remove microorganisms from the collected saliva.

Preferably, the detection module may be configured to change a pH value of the collected saliva into pH7.

Preferably, the detection module may be configured to add a buffer solution to mix the buffer solution with the collected saliva.

Preferably, the analysis module may be configured to analyze whether there is a fertile window through an antigen-antibody reaction, in which an immune reaction of the analysis module is the antigen-antibody reaction between the hormone detected from the collected saliva and an antibody.

More preferably, the hormone may sequentially react with the antibody, promote a color formation, and amplify the promoted color formation.

Further, in order to achieve the above objects, a fertile window detection method using a fertile window detection kit using saliva is configured to include: (a) collecting, by a collection module, saliva of a user; (b) detecting, by a detection module, a hormone from the saliva after removing mucus glycoproteins from the collected saliva as a pre-treatment process for detecting the hormone from the saliva collected from the collection module of step (a); (c) analyzing, by an analysis module, whether there is a fertile window by using an immune reaction of the hormone detected from the detection module of step (b); and (d) displaying, by a display module, information to confirm whether there is a fertile window based on the analysis result of the analysis module of step (c).

Preferably, the hormone may include a luteinizing hormone.

Preferably, the mucus glycoproteins may include mucosal glycoproteins.

Preferably, the removal of the mucus glycoproteins may be obtained by decomposing glucoproteins within the collected saliva.

More preferably, mucus may be removed to purify the saliva after decomposing the glucoproteins.

Preferably, the detection module may be configured to remove microorganisms from the collected saliva.

Preferably, the detection module may be configured to change a pH value of the collected saliva into pH7.

Preferably, the detection module may be configured to add a buffer solution to mix the buffer solution with the collected saliva.

Preferably, the analysis module may be configured to analyze whether there is a fertile window through an antigen-antibody reaction, in which an immune reaction of the analysis module is the antigen-antibody reaction between the hormone detected from the collected saliva and an antibody.

More preferably, the hormone may sequentially react with the antibody, promote a color formation, and amplify the promoted color formation.

### Advantageous Effects of the Invention

According to a breast cancer detection kit using saliva and a breast cancer detection method using the kit, which are presented in the present invention, the breast cancer detection kit is configured to include: a collection module configured to collect saliva of a user; a detection module configured to detect a breast cancer marker from the saliva after removing mucus glycoproteins from the collected saliva as a pre-treatment process for detecting the breast cancer marker from the saliva collected from the collection module; and an analysis module configured to analyze a presence of breast cancer by using an immune reaction of the breast cancer marker detected from the detection module, wherein the breast cancer marker detected from the saliva includes at least one of Lysine (409.2312 m/z), Threonine (131.1174 m/z), Glutamic acid, Tyrosine, Piperideine, Valine, and Glycine (437.7442 m/z), wherein a process for removing mucus glycoproteins from the collected saliva is performed as a pre-treatment process for detecting the breast cancer marker, and wherein the analysis module is configured to analyze the presence of breast cancer through a reaction with an antibody which performs an antigen-antibody reaction with the breast cancer marker, thereby more accurately detecting the presence of breast cancer than a conventional technique for detecting breast cancer.

Further, according to the present invention, the breast cancer marker sequentially reacts with an antibody, promotes a color formation and amplifies the promoted color formation, and the breast cancer detection kit is configured to include a display module which displays information to confirm the presence of breast cancer based on the analysis result of the analysis module, wherein the presence of breast cancer may be confirmed with naked eyes without a particular need for a separate equipment, and wherein the breast cancer detection kit costs less than a conventional technique for detecting breast cancer, may be easily portable, may be popularized, and may detect a woman's breast cancer at an early stage to be treated.

Meanwhile, according to a fertile window detection kit using saliva and a fertile window detection method using the kit, which are presented in the present invention, the fertile window detection kit is configured to include: a collection module configured to collect saliva of a user; a detection module configured to detect a hormone from the saliva collected from the collection module; and an analysis module configured to analyze whether there is a fertile window by using an immune reaction of the hormone detected from the detection module, wherein the analysis module is configured to analyze whether there is a fertile window through an antigen-antibody reaction as the antigen-antibody reaction between the hormone and the antibody, thereby resolving inconveniences of limitations on time and place in a conventional technology for confirming whether there is a fertile window through urine, and solving problems of the conventional technique for determining the structure of saliva through observation, in which saliva takes a certain amount of time to dry and the observation, itself, of spit can arouse visual discomfort, in order to confirm the salt crystals of saliva or the structural shape of saliva.

Further, according to the present invention, the hormone collected from the collection module includes a luteinizing hormone and a process for removing mucus to purify the saliva after decomposing the glucoproteins within the collected saliva is performed as a pre-treatment process for detecting the hormone, which removes mucus glycoproteins from the collected saliva, and a process for changing a pH value of the collected saliva into pH7 and adding a buffer solution to mix the buffer solution with the collected saliva is performed in order to remove microorganisms and facilitate an antigen-antibody reaction, thereby increasing the accuracy of detecting and analyzing the hormone, and more accurately detecting whether there is a fertile window than a conventional technique for detecting the fertile window.

In addition, the hormone sequentially reacts with an antibody, promotes a color formation and amplifies the promoted color formation, and the fertile window detection kit is configured to include a display module which displays information to confirm whether there is a fertile window based on the analysis result of the analysis module, wherein whether there is a fertile window may be confirmed with naked eyes without a particular need for a separate equipment and may be quickly confirmed without a need for additional time for saliva to get dry compared to a conventional technique for detecting a fertile window, and thus the fertile window detection kit may be small-sized, may be easily portable and may be popularized.

### DESCRIPTION OF THE DRAWINGS

FIG. 1 is a view showing a test result of a conventional mammography.

FIG. 2 is a view showing a test result of a conventional breast ultrasonography.

FIG. 3 is a view for explaining a woman's menstrual cycle.

FIG. 4 is a view showing a conventional ovulation tester using urine.

FIG. 5 is a view showing a conventional portable ovulation tester using saliva.

FIG. 6 is a view for explaining a detection principle of a conventional portable ovulation tester using saliva.

FIG. 7 is a view showing an entire configuration of a breast cancer detection kit using saliva according to one embodiment of the present invention.

FIG. 8 is a view for explaining a general antigen-antibody reaction.

FIG. 9 is a view for explaining a color formation reaction through a general antigen-antibody reaction.

FIG. 10 is a flowchart showing a breast cancer detection method using a breast cancer detection kit using saliva according to one embodiment of the present invention.

FIG. 11 is a view showing an entire configuration of a fertile window detection kit using saliva according to one embodiment of the present invention.

FIG. 12 is a flowchart showing a fertile window detection method using a fertile window detection kit using saliva according to one embodiment of the present invention.

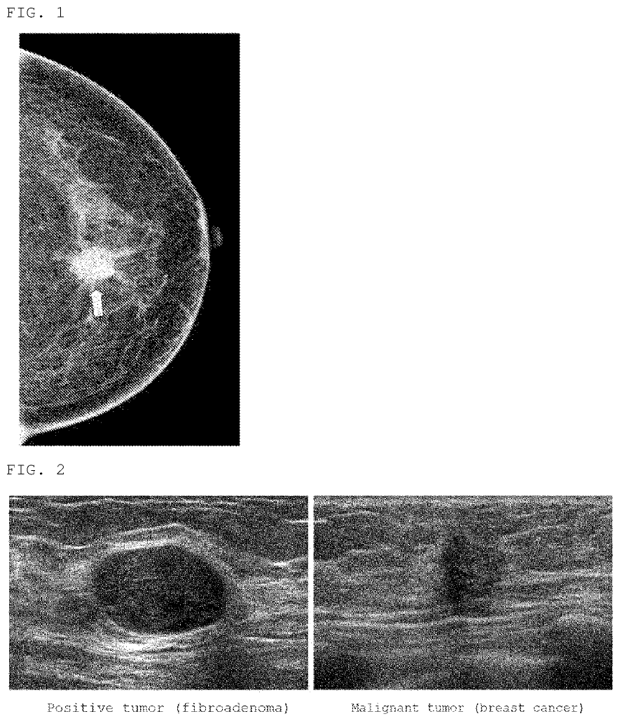

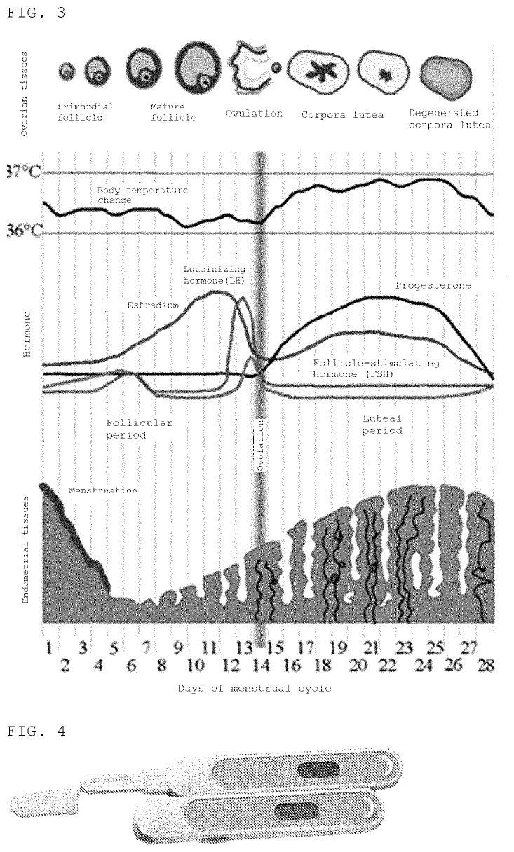

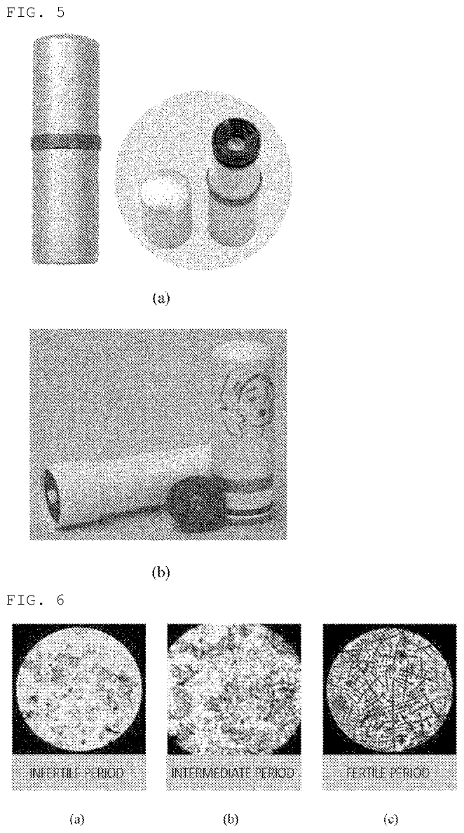

### DESCRIPTION OF REFERENCE NUMERALS

10: Breast cancer detection kit using saliva according to one embodiment of the present invention
10′: Fertile window detection kit using saliva according to one embodiment of the present invention
100, 100′: Collection module
200, 200′: Detection module
300, 300′: Analysis module
400, 400′: Display module

### BEST MODE

Hereinafter, the preferred embodiments will be described in detail such that those skilled in the art to which the present invention pertains may easily carry out the present invention with reference to the accompanying drawings. In the following description of the present invention, a detailed description of known functions and configurations incorporated herein will be omitted when it may make the subject matter of the present invention unnecessarily unclear.

In addition, when a part is referred to as being “connected” to another part, it includes not only “directly connected”, but also “indirectly connected” with another element interposed therebetween throughout the specification. In addition, when a component is referred to as “including” a certain element, it is to be understood that the component may further include other elements as well without excluding the other elements unless specifically defined otherwise.

FIG. 7 is a view showing an entire configuration of a breast cancer detection kit using saliva according to one embodiment of the present invention. As shown in FIG. 7, the breast cancer detection kit 10 using saliva according to one embodiment of the present invention is configured to include: a collection module 100 configured to collect saliva of a user; a detection module 200 configured to detect a breast cancer marker from the saliva after removing mucus glycoproteins from the collected saliva as a pre-treatment process for detecting the breast cancer marker from the saliva collected from the collection module 100; an analysis module 300 configured to analyze a presence of breast cancer by using an immune reaction of the breast cancer marker detected from the detection module 200; and a display module 400 configured to display information to confirm the presence of breast cancer based on the analysis result of the analysis module 300.

The collection module 100 plays a role to collect saliva from a user, and the detection module 200 plays a role to detect a breast cancer marker from the saliva collected from the collection module 100. The breast cancer marker collected may include at least one of Lysine (409.2312 m/z), Threonine (131.1174 m/z), Glutamic acid, Tyrosine, Piperideine, Valine, and Glycine (437.7442 m/z).

The detection module 200 may be configured to remove mucus glycoproteins from the collected saliva as a pre-treatment process for detecting the breast cancer marker, and the mucus glycoproteins may include mucosal glycoproteins. Specifically, the detection module may be configured to sequentially decompose glycoproteins by using a syringe filter, remove mucus, and remove mucus glycoproteins to purify saliva, in order to measure the proteins contained in the saliva. The detection module may be configured to remove microorganisms from the collected saliva after removing mucus glycoproteins therefrom. Further, the detection module 200 may be configured to change a pH value of the collective saliva from pH5-6 into pH7. Such change aims at creating an environment in which an immune reaction to be described below, that is, an antigen-antibody reaction may be smoothly performed. According to embodiments, a buffer solution may be added to mix with the collected saliva, thereby facilitating the immune reaction.

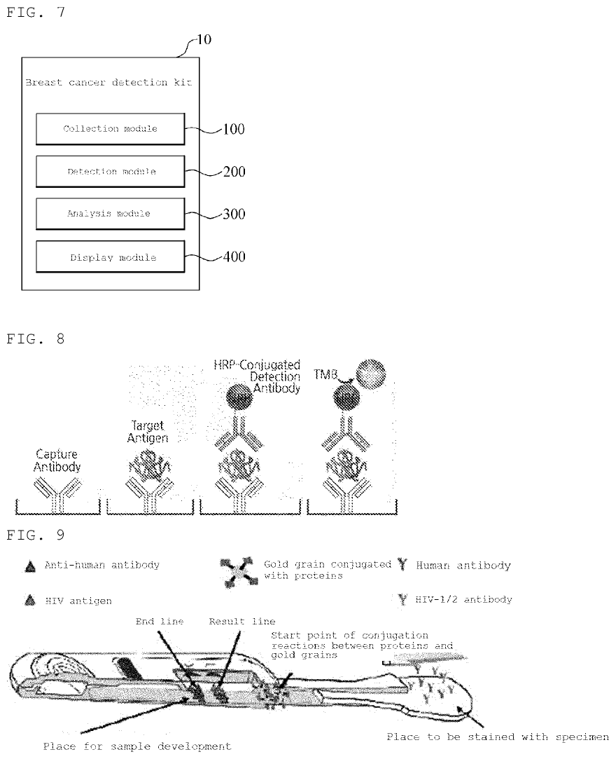

The analysis module 300 plays a role to analyze a presence of breast cancer by using an immune reaction of the breast cancer marker detected from the detection module 200. FIG. 8 is a view for explaining a general antigen-antibody reaction. It may be confirmed that a shape of a capture antibody, a shape of the reaction between the antibody and a target antigen, a shape of the reaction with an antibody conjugated with horseradish peroxidase (HRP), and a shape of the color formation with the antibody conjugated with HRP by using TMB are shown in order from the left. In the breast cancer detection kit 10 using saliva according to the present invention, an immune reaction of the analysis module 300 is an antigen-antibody reaction between the breast cancer marker detected from the collected saliva and the antibody as shown in the reaction of FIG. 8, in which a presence of breast cancer may be analyzed through such antigen-antibody reaction. According to embodiments, the detection module 200 may be configured in such a way that the purified saliva passes through a sheet and a breast cancer marker having passed through the sheet reacts with an antibody to promote a color formation as a result of the reaction and amplify the promoted color formation.

FIG. 9 is a view for explaining a color formation reaction through a general antigen-antibody reaction, in which test results are confirmed by using an antigen-antibody color formation reaction. With regard to the color formation reaction, there is a difference between pregnancy or ovulation and HIV or hepatitis C. In case of pregnancy or ovulation, an antigen hCG or LH in urine, an antibody conjugated with gold grains, and an antibody in a result line are subjected to a double reaction to show a color formation reaction. In contrast, in case of HIV or hepatitis C, an antibody produced in body against an invading exogenous antigen and an antigen in a result line are subjected to a reaction to show a color formation reaction and confirm the result. The breast cancer detection kit 10 using saliva according to the present invention is also configured to carry out a color formation through the color formation reaction of the antibody as described in FIG. 9, and promote and amplify such color formation.

The display module 400 plays a role to display information such that a user may confirm the presence of breast cancer based on the analysis result of the analysis module 300. In other words, the analysis module 300 is configured to analyze the presence of breast cancer from the results of color formation, promotion and amplification through an immune reaction. The display module 400 is configured to display information such that a user may recognize whether the analysis result of the analysis module 300 shows the breast cancer or not. Each of the collection module 100, the detection module 200, the analysis module 300 and the display module 400 may be configured in a separate way, but preferably may be all disposed in one kit. In this case, the kit has an advantage of being small-sized, more portable and thus having more popularity in use.

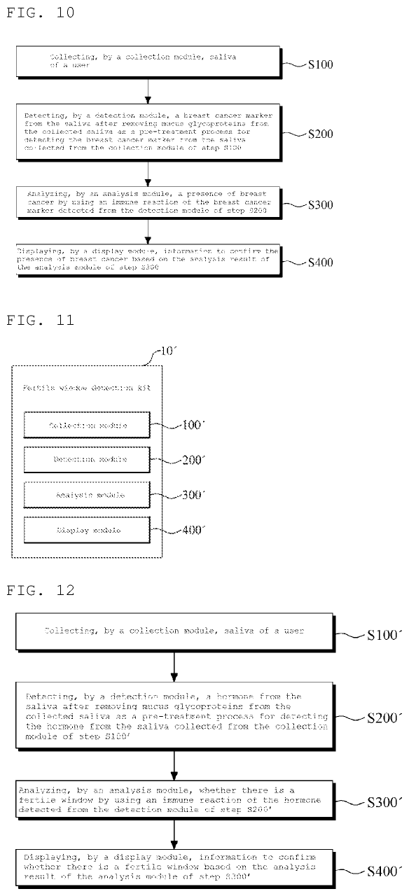

FIG. 10 is a flowchart showing a breast cancer detection method using a breast cancer detection kit using saliva according to one embodiment of the present invention. As shown in FIG. 10, the breast cancer detection method using a breast cancer detection kit 10 using saliva according to one embodiment of the present invention is configured to include: collecting, by a collection module 100, saliva of a user (S100); detecting, by a detection module 200, a breast cancer marker from the saliva after removing mucus glycoproteins from the collected saliva as a pre-treatment process for detecting the breast cancer marker from the saliva collected from the collection module of step S100 (S200); analyzing, by an analysis module 300, a presence of breast cancer by using an immune reaction of the breast cancer marker detected from the detection module 200 of step S200 (S300); and displaying, by a display module 400, information to confirm the presence of breast cancer based on the analysis result of the analysis module 300 of step S300 (S400).

The details of each of the steps have been fully described above with regard to the breast cancer detection kit 10 using saliva according to one embodiment of the present invention, and thus the detailed description thereof will be omitted below.

Meanwhile, FIG. 11 is a view showing an entire configuration of a fertile window detection kit using saliva according to one embodiment of the present invention. As shown in FIG. 11, the fertile window detection kit 10′ using saliva according to one embodiment of the present invention is configured to include: a collection module 100′ configured to collect saliva of a user; a detection module 200′ configured to detect a hormone from the saliva after removing mucus glycoproteins from the collected saliva as a pre-treatment process for detecting the hormone from the saliva collected from the collection module 100′; an analysis module 300′ configured to analyze whether there is a fertile window by using an immune reaction of the hormone detected from the detection module 200′; and a display module 400′ configured to display information to confirm whether there is a fertile window based on the analysis result of the analysis module 300′.

The collection module 100′ plays a role to collect saliva from a user, and the detection module 200′ plays a role to detect a hormone from the saliva collected from the collection module 100′. The collected hormone may include a luteinizing hormone.

The detection module 200′ may be configured to remove mucus glycoproteins from the collected saliva as a pre-treatment process for detecting the hormone, and the mucus glycoproteins may include mucosal glycoproteins. Specifically, the detection module may be configured to sequentially decompose glycoproteins by using a syringe filter, remove mucus, and remove mucus glycoproteins to purify saliva, in order to measure the proteins contained in the saliva. The detection module may be configured to remove microorganisms from the collected saliva after removing mucus glycoproteins therefrom. Further, the detection module 200′ may be configured to change a pH value of the collective saliva from pH5-6 into pH7. Such change aims at creating an environment in which an immune reaction to be described below, that is, an antigen-antibody reaction may be smoothly performed. According to embodiments, a buffer solution may be added to mix with the collected saliva, thereby facilitating the immune reaction.

The analysis module 300′ plays a role to analyze whether there is a fertile window by using an immune reaction of the hormone detected from the detection module 200′. As described above, FIG. 8 is a view for explaining a general antigen-antibody reaction. It may be confirmed that a shape of a capture antibody, a shape of the reaction between the antibody and a target antigen, a shape of the reaction with an antibody conjugated with horseradish peroxidase (HRP), and a shape of the color formation with the antibody conjugated with HRP by using TMB are shown in order from the left. In the fertile window detection kit 10′ using saliva according to the present invention, an immune reaction of the analysis module 300′ is an antigen-antibody reaction between the hormone detected from the collected saliva and the antibody as shown in the reaction of FIG. 8, in which whether there is a fertile window may be analyzed through such antigen-antibody reaction. According to embodiments, the detection module 200′ may be configured in such a way that the purified saliva passes through a sheet and a hormone having passed through the sheet reacts with an antibody to promote a color formation as a result of the reaction and amplify the promoted color formation.

As described above, FIG. 9 is a view for explaining a color formation reaction through a general antigen-antibody reaction, in which test results are confirmed by using an antigen-antibody color formation reaction. With regard to the color formation reaction, there is a difference between pregnancy or ovulation and HIV or hepatitis C. In case of pregnancy or ovulation, an antigen hCG or LH in urine, an antibody conjugated with gold grains, and an antibody in a result line are subjected to a double reaction to show a color formation reaction. In contrast, in case of HIV or hepatitis C, an antibody produced in body against an invading exogenous antigen and an antigen in a result line are subjected to a reaction to show a color formation reaction and confirm the result. The fertile window detection kit 10′ using saliva according to the present invention is also configured to carry out a color formation through the color formation reaction of the antibody as described in FIG. 9, and promote and amplify such color formation.

The display module 400′ plays a role to display information such that a user may confirm whether there is a fertile window based on the analysis result of the analysis module 300′. In other words, the analysis module 300′ is configured to analyze whether there is a fertile window from the results of color formation, promotion and amplification through an immune reaction. The display module 400′ is configured to display information such that a user may recognize whether there is a fertile window based on the analysis result of the analysis module 300′. Each of the collection module 100′, the detection module 200′, the analysis module 300′ and the display module 400′ may be configured in a separate way, but preferably may be all disposed in one kit. In this case, the kit has an advantage of being small-sized, more portable and thus having more popularity in use.

FIG. 12 is a flowchart showing a fertile window detection method using a fertile window detection kit using saliva according to one embodiment of the present invention. As shown in FIG. 12, the fertile window detection method using a fertile window detection kit 10′ using saliva according to one embodiment of the present invention is configured to include: collecting, by a collection module 100′, saliva of a user (S100′); detecting, by a detection module 200′, a hormone from the saliva after removing mucus glycoproteins from the collected saliva as a pre-treatment process for detecting the hormone from the saliva collected from the collection module of step S100′ (S200′); analyzing, by an analysis module 300′, whether there is a fertile window by using an immune reaction of the hormone detected from the detection module 200′ of step S200′ (S300′); and displaying, by a display module 400′, information to confirm whether there is a fertile window based on the analysis result of the analysis module 300′ of step S300′ (S400′).

The details of each of the steps have been fully described above with regard to the fertile window detection kit 10′ using saliva according to one embodiment of the present invention, and thus the detailed description thereof will be omitted below.

The present invention as described above may be subjected to various modifications or applications by those skilled in the art to which the present invention pertains, and the technical scope of the present invention may be determined by the patent claims to be described below.

1. A breast cancer detection kit using saliva, the breast cancer detection kit comprising:
a collection module configured to collect saliva of a user;
a detection module configured to detect a breast cancer marker from the saliva after removing mucus glycoproteins from the collected saliva as a pre-treatment process for detecting the breast cancer marker from the saliva collected from the collection module;
an analysis module configured to analyze a presence of breast cancer by using an immune reaction of the breast cancer marker detected from the detection module; and
a display module configured to display information to confirm the presence of breast cancer based on the analysis result of the analysis module.

2. The breast cancer detection kit of claim 1, wherein the breast cancer marker comprises at least one of Lysine (409.2312 m/z), Threonine (131.1174 m/z), Glutamic acid, Tyrosine, Piperideine, Valine, and Glycine (437.7442 m/z).

3. The breast cancer detection kit of claim 1, wherein the mucus glycoproteins comprise mucosal glycoproteins.

4. The breast cancer detection kit of claim 1, wherein the removal of the mucus glycoproteins is obtained by decomposing glucoproteins within the collected saliva.

5. The breast cancer detection kit of claim 4, wherein mucus is removed to purify the saliva after decomposing the glucoproteins.

6. The breast cancer detection kit of claim 1, wherein the detection module is configured to remove microorganisms from the collected saliva.

7. The breast cancer detection kit of claim 1, wherein the detection module is configured to change a pH value of the collected saliva into pH7.

8. The breast cancer detection kit of claim 1, wherein the detection module is configured to add a buffer solution to mix the buffer solution with the collected saliva.

9. The breast cancer detection kit of claim 1, wherein the analysis module is configured to analyze the presence of breast cancer through an antigen-antibody reaction, in which an immune reaction of the analysis module is the antigen-antibody reaction between the breast cancer marker detected from the collected saliva and an antibody.

10. The breast cancer detection kit of claim 9, wherein the breast cancer marker sequentially reacts with the antibody, promotes a color formation, and amplifies the promoted color formation.

11. A breast cancer detection method using a breast cancer detection kit using saliva, the breast cancer detection method comprising:
(a) collecting, by a collection module, saliva of a user;
(b) detecting, by a detection module, a breast cancer marker from the saliva after removing mucus glycoproteins from the collected saliva as a pre-treatment process for detecting the breast cancer marker from the saliva collected from the collection module of step (a);
(c) analyzing, by an analysis module, a presence of breast cancer by using an immune reaction of the breast cancer marker detected from the detection module of step (b); and
(d) displaying, by a display module, information to confirm the presence of breast cancer based on the analysis result of the analysis module of step (c).

12. The breast cancer detection method of claim 11, wherein the breast cancer marker comprises at least one of Lysine (409.2312 m/z), Threonine (131.1174 m/z), Glutamic acid, Tyrosine, Piperideine, Valine, and Glycine (437.7442 m/z).

13. The breast cancer detection method of claim 11, wherein the mucus glycoproteins comprise mucosal glycoproteins.

14. The breast cancer detection method of claim 11, wherein the removal of the mucus glycoproteins is obtained by decomposing glucoproteins within the collected saliva.

15. The breast cancer detection method of claim 14, wherein mucus is removed to purify the saliva after decomposing the glucoproteins.

16. The breast cancer detection method of claim 11, wherein the detection module is configured to remove microorganisms from the collected saliva.

17. The breast cancer detection method of claim 11, wherein the detection module is configured to change a pH value of the collected saliva into pH7.

18. The breast cancer detection method of claim 11, wherein the detection module is configured to add a buffer solution to mix the buffer solution with the collected saliva.

19. The breast cancer detection method of claim 11, wherein the analysis module is configured to analyze the presence of breast cancer through an antigen-antibody reaction, in which an immune reaction of the analysis module is the antigen-antibody reaction between the breast cancer marker detected from the collected saliva and an antibody.

20. The breast cancer detection method of claim 19, wherein the breast cancer marker sequentially reacts with the antibody, promotes a color formation, and amplifies the promoted color formation.

21. A fertile window detection kit using saliva, the fertile window detection kit comprising:
a collection module configured to collect saliva of a user;
a detection module configured to detect a hormone from the saliva after removing mucus glycoproteins from the collected saliva as a pre-treatment process for detecting the hormone from the saliva collected from the collection module;
an analysis module configured to analyze whether there is a fertile window by using an immune reaction of the hormone detected from the detection module; and
a display module configured to display information to confirm whether there is a fertile window based on the analysis result of the analysis module.

22. The fertile window detection kit of claim 21, wherein the hormone comprises a luteinizing hormone.

23. The fertile window detection kit of claim 21, wherein the mucus glycoproteins comprise mucosal glycoproteins.

24. The fertile window detection kit of claim 21, wherein the removal of the mucus glycoproteins is obtained by decomposing glucoproteins within the collected saliva.

25. The fertile window detection kit of claim 24, wherein mucus is removed to purify the saliva after decomposing the glucoproteins.

26. The fertile window detection kit of claim 21, wherein the detection module is configured to remove microorganisms from the collected saliva.

27. The fertile window detection kit of claim 21, wherein the detection module is configured to change a pH value of the collected saliva into pH7.

28. The fertile window detection kit of claim 21, wherein the detection module is configured to add a buffer solution to mix the buffer solution with the collected saliva.

29. The fertile window detection kit of claim 21, wherein the analysis module is configured to analyze whether there is a fertile window through an antigen-antibody reaction, in which an immune reaction of the analysis module is the antigen-antibody reaction between the hormone detected from the collected saliva and an antibody.

30. The fertile window detection kit of claim 29, wherein the hormone sequentially reacts with the antibody, promotes a color formation, and amplifies the promoted color formation.

31. A fertile window detection method using a fertile window detection kit using saliva, the fertile window detection method comprising:
(a) collecting, by a collection module, saliva of a user;
(b) detecting, by a detection module, a hormone from the saliva after removing mucus glycoproteins from the collected saliva as a pre-treatment process for detecting the hormone from the saliva collected from the collection module of step (a);
(c) analyzing, by an analysis module, whether there is a fertile window by using an immune reaction of the hormone detected from the detection module of step (b); and
(d) displaying, by a display module, information to confirm whether there is a fertile window based on the analysis result of the analysis module of step (c).

32. The fertile window detection method of claim 31, wherein the hormone comprises a luteinizing hormone.

33. The fertile window detection method of claim 31, wherein the mucus glycoproteins comprise mucosal glycoproteins.

34. The fertile window detection method of claim 31, wherein the removal of the mucus glycoproteins is obtained by decomposing glucoproteins within the collected saliva.

35. The fertile window detection method of claim 34, wherein mucus is removed to purify the saliva after decomposing the glucoproteins.

36. The fertile window detection method of claim 31, wherein the detection module is configured to remove microorganisms from the collected saliva.

37. The fertile window detection method of claim 31, wherein the detection module is configured to change a pH value of the collected saliva into pH7.

38. The fertile window detection method of claim 31, wherein the detection module is configured to add a buffer solution to mix the buffer solution with the collected saliva.

39. The fertile window detection method of claim 31, wherein the analysis module is configured to analyze whether there is a fertile window through an antigen-antibody reaction, in which an immune reaction of the analysis module is the antigen-antibody reaction between the hormone detected from the collected saliva and an antibody.

40. The fertile window detection method of claim 39, wherein the hormone sequentially reacts with the antibody, promotes a color formation, and amplifies the promoted color formation.
